(19) United States
(12) Patent Application Publication
(10) Pub. No.: US 2016/0084815 A1
(43) Pub. Date: Mar. 24, 2016

(54) METHODS AND SYSTEMS FOR QUANTIFYING THE GRADE OF PETROLEUM OIL BASED ON FLUORESCENCE

(71) Applicant: AXURE TECHNOLOGIES S.A., Bogota (CO)

(72) Inventors: Raul CUERO RENGIFO, Cypress, TX (US); Jhon Henry TRUJILLO MONTENEGRO, Cali (CO); Jennifer Melissa RUSSI CASTILLO, Manizales (CO)

(73) Assignee: AXURE TECHNOLOGIES S.A., Bogota (CO)

(21) Appl. No.: 14/892,367

(22) PCT Filed: May 21, 2014
(86) PCT No.: PCT/US14/38890

§ 371 Date: Nov. 19, 2015

Related U.S. Application Data
(60) Provisional application No. 61/826,259, filed on May 22, 2013.

(51) Int. Cl. G01N 33/22; G01N 21/64

(52) U.S. Cl. CPC G01N 33/22; G01N 21/645; G01N 21/643; G01N 2021/6484

(57) ABSTRACT

Systems and methods evaluate the grade or API gravity of petroleum. The methods and systems disclosed herein overcome many of the difficulties commonly associated with petroleum exploration. The methods and systems include collecting fluorescence emission spectra of soil samples and inputting these into an artificial neural network, which is able to rapidly and accurately assess and classify data from multiple soil samples. Additionally, the methods and systems can be useful in identifying other compounds related to or commonly found together with petroleum, including hydrocarbons and aromatic compounds.

### CROSS REFERENCE TO RELATED APPLICATION

This application claims priority upon U.S. Provisional Application Ser. No. 61/826,259, filed May 22, 2013. The application is hereby incorporated by reference in its entirety for all of its teachings.

### BACKGROUND

Oil exploration is a process which is not only costly and uncertain but also time-consuming, technology-intensive, and labor-intensive. Typically, a large capital investment is required. Many oil exploration techniques involve targeting traces of petroleum which are believed to be connected to, or geographically near, larger petroleum reserves.

Existing methods of oil exploration involve satellite imaging to compare and analyze features of geography and/or terrain, seismic and/or magnetic surveys, and combination laser/optical sensor devices such as the Rapid Optical Screening Tool (ROST). However, these technologies involve the use of expensive and specialized equipment, and are often unable to determine the type or grade of petroleum present in a sample. Further, a ROST device is typically mounted on a truck or other vehicle and may not be useful for petroleum exploration in remote areas lacking roads.

It would therefore be desirable to develop an improved method and/or system for detecting and subsequently categorizing petroleum oil in soil samples. Such a method and system are objects of the present invention.

Additionally, a detection method and system such as those described herein may find uses in applications such as environmental testing, clean-up after oil spills, and the like.

### SUMMARY

Described herein are systems and methods for evaluating the grade or API gravity of petroleum. The methods and systems disclosed herein overcome many of the difficulties commonly associated with petroleum exploration. The methods and systems include collecting fluorescence emission spectra of soil samples and inputting these into an artificial neural network, which is able to rapidly and accurately assess and classify data from multiple soil samples. Additionally, the methods and systems can be useful in identifying other compounds related to or commonly found together with petroleum, including hydrocarbons and aromatic compounds.

These and other aspects, features, and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a block diagram illustrating an example of the network environment for the petroleum detection system described herein.

FIG. 2 is a block diagram illustrating an example of a server utilizing the petroleum detection system described herein, as shown in FIG. 1.

FIG. 3 is a flow chart illustrating an example of a server utilizing the petroleum detection system described herein utilized by the server, as shown in FIG. 2.

FIG. 4 is a flow chart illustrating an example of the operation of the library construction process on the server that is utilized in the petroleum detection system described herein, as shown in FIGS. 2-3.

FIG. 5 is a flow chart illustrating an example of the operation of the petroleum analysis process on the server that is utilized in the petroleum detection system described herein, as shown in FIGS. 2-3.

FIG. 6 shows an exemplary reactor for sediment conditions used in a controlled-environment simulation.

FIG. 7 shows a sample system with UV lamp and optical fiber.

### DETAILED DESCRIPTION

The present invention may be understood more readily by reference to the following detailed description of the invention taken in connection with the accompanying drawing figures, which form a part of this disclosure. It is to be understood that this invention is not limited to specific devices, compounds, compositions, methods, conditions, or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

In this specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “a transition metal” includes two or more metals, and the like.

“Optional” or “optionally” means that the subsequently described event or circumstance can or cannot occur, and that the description includes instances where the event or circumstance occurs and instances where it does not.

As used herein, the term “about” is used to provide flexibility to a numerical range endpoint by providing that a given value may be “a little above” or “a little below” the endpoint without affecting the desired result.

As used herein, “petroleum” or “crude oil” or “oil” refers to a naturally-occurring fossil fuel that includes hydrocarbons and/or other organic molecules, optionally including sulfur compounds, and most often found beneath the Earth's surface. In some cases, petroleum is also detectable in surface-level soil. Petroleum and/or crude oil can be refined into multiple other products including gasoline and other fuels, as well as asphalt, components of plastics, etc.

“Fluorescence” is a phenomenon in which light is emitted by a substance that has absorbed light. Light emitted by fluorescence has, in most cases, a longer wavelength than the light that was absorbed. Without wishing to be bound by theory, electrons in a fluorescent material are excited by the absorbed light; de-excitation of the electrons and emission of photons occur nearly spontaneously with absorption. In some aspects, the intensity of fluorescence emission is measured at only one wavelength. In other aspects, a fluorescence spectrum that includes the intensities of fluorescence emissions at a plurality of wavelengths may be recorded.

As used herein, the term “library” may have two different meanings, which will be distinguishable by the context in which they are used. In one aspect, a “library” is a set of defined functions or code that can be accessed by a computer program; in this sense, a library function can be called by different software programs without having to be rewritten or copied into each piece of software. In an alternative aspect, a “library” is a collection or database of information about samples (e.g. soil samples potentially containing petroleum) to be analyzed. In this aspect, a piece of computer software may analyze samples and add information about said samples to the library, or may search the library to compare known samples (whose information is contained within the library) to unknown samples in order to classify said unknown samples.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an illustration, a numerical range of “about 1 to about 5” should be interpreted to include not only the explicitly recited values of about 1 to about 5, but also to include individual values and sub-ranges within the indicated range. Thus, included in this numerical range are individual values such as 2, 3, and 4, and sub ranges such as from 1-3, from 2-4, and from 3-5, etc., as well as 1, 2, 3, 4, and 5, individually. The same principle applies to ranges reciting only one numerical value as a minimum or a maximum. Furthermore, such an interpretation should apply regardless of the breadth of the range or the characteristics being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. For example, if a grade of petroleum is disclosed and discussed and a number of different transition metals are discussed, each and every combination and permutation of petroleum grade and transition metal that is possible is specifically contemplated unless specifically indicated to the contrary. For example, if a class of molecules A, B, and C are disclosed, as well as a class of molecules D, E, and F, and an example of a combination A+D is disclosed, then even if each is not individually recited, each is individually and collectively contemplated. Thus, in this example, each of the combinations A+E, A+F, B+D, B+E, B+F, C+D, C+E, and C+F, are specifically contemplated and should be considered disclosed from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered disclosed from disclosure of A, B, and C; D, E, and F; and the example combination of A+D. This concept applies to all aspects of this disclosure including, but not limited to, steps in methods of making and using the disclosed compositions. Thus, if there are a variety of additional steps that can be performed with any specific embodiment or combination of embodiments of the disclosed methods, and that each such combination is specifically contemplated and should be considered disclosed.

As used herein, the “American Petroleum Institute gravity” or “API gravity” refers to a measure of the density of a petroleum liquid relative to water. API gravity is represented by a number, for example, from about 0 to about 48. The “grade” of a petroleum product is determined based on its API gravity. Heavy crude oil has an API gravity ranging from about 10.0 to about 22.3 and medium crude oil has an API gravity ranging from about 22.3 to about 31.1, while light crude oil has an API gravity higher than 31.1. Extra heavy crude oil is also known, and has an API gravity below about 10.0. API gravity may be calculated from the density and/or specific gravity of a petroleum product by any of the following methods, or by similar or related methods: ASTM D1298, ASTM D4052, ASTM D1250, or ASTM D287.

### Neural Network

A “neural network” as used herein refers to an artificial neural network, which is a computerized implementation of a mathematical model and which represents one approach to machine learning. A neural network is made up of several layers of artificial neurons, or “nodes,” and always contains an input layer of nodes that receives information as well as an output layer that provides a weighted sum of the inputs. A neural network can include one or more hidden layers containing weights to be applied to the information supplied to them; these weights are determined by the network itself through training on sample data sets. A neural network also includes an activation function that converts weighted input to output. In some aspects, neural networks are used for classification of inputs, including pattern recognition and feature extraction. In other aspects, neural networks can be used to remove noise from a signal or to predict outcomes based on extrapolation of historical data. Without wishing to be bound by theory, neural networks function on their own based only on sample inputs and are able to generalize, or produce outputs for inputs they have not previously encountered. Neural networks are also useful when the relationship between outputs and inputs is not clearly understood, and/or when input data sets are particularly large.

As used herein, an “input vector” is a list of data items passed to a neural network. In one aspect, the list of data items may be a fluorescence emission spectrum consisting of a series of fluorescence intensities at a corresponding series of wavelengths.

As used herein, an “output vector” is a single value or a series of values resulting from the analysis of inputs by a neural network. In one aspect, the output vector is a single number which can have a range of possible values corresponding to specific categories (e.g. API gravity). In another aspect, the output vector is two or more numbers with discrete values (e.g. 0, 1, etc.) indicating the presence or absence of a specific component from a sample. In a further aspect, 0 indicates absence of a metal from a petroleum sample and 1 indicates presence of that metal in a petroleum sample. In a related aspect, the metals can be vanadium and/or nickel.

A “hidden layer vector” is a vector intermediate in size between an input vector and an output vector. The hidden layer vector contains weights which are applied to input values in a neural network. In one aspect, these weights are adjusted as the neural network undergoes training.

In some aspects, information about samples suspected of containing petroleum and/or transition metals can be collected by a device such as a camera or a spectrometer. In further aspects, this information can be processed by a computer as described below and in the accompanying drawings. After, or as part of, this processing by a computer, the information about the samples and materials to be analyzed for petroleum can be input into an artificial neural network. In some aspects, the use of a neural network can be an alternative to statistical analysis, particularly when the relationship between sample information (input) and petroleum grade (output) is not fully understood. In one aspect, the neural network described herein may have a single output node identifying a predicted class to which a sample belongs. In a further aspect, the predicted class may be API gravity. In another aspect, the neural network described herein may have two or more output nodes representing different classes. In a further aspect, the classes may be assigned to particular transition metals. In a still further aspect, the transition metals may be nickel and vanadium.

In one aspect, the neural network architecture is selected from the group including, but not limited to, an ADALINE Neural Network, an Adaptive Resonance Theory 1 (ART1) Neural Network, a Bidirectional Associative Memory (BAM) Neural Network, a Boltzmann Machine Neural Network, a Counterpropagation Neural Network (CPN), an Elman Recurrent Neural Network, a Feedforward Neural Network (also known as a Perceptron), a Hopfield Neural Network, a Jordan Recurrent Neural Network, a Neuroevolution of Augmenting Topologies (NEAT) Neural Network, a Radial Basis Function Network, a Recurrent Self-Organizing Map (RSOM) Neural Network, or a Self-Organizing Map Neural Network (also known as a Kohonen Neural Network). In a further aspect, a feedforward or perceptron neural network is used.

As used herein, a “feedforward” neural network is a neural network in which information flows in only one direction; that is, from an input layer to an output layer or from an input layer to one or more hidden layers to an output layer. In one aspect, the feedforward neural network includes one hidden layer.

In some aspects, the neural network described herein is subjected to a training technique prior to analysis of unknown samples. In one aspect, the training technique is a supervised training technique, wherein the neural network is provided with input for a series of samples and with the corresponding output expected for said samples. In one aspect, the output can be a value corresponding to a particular grade of petroleum (i.e., API gravity). In another aspect, the training technique is an unsupervised training technique, wherein the neural network is provided with input for a series of samples but is not provided with any expected output. In this aspect, the neural network system itself identifies and responds to patterns and features of the input. In a further aspect, the training technique is a supervised training technique.

In another aspect, the neural network training technique is a backpropagation technique. As used herein, a “backpropagation” technique is a supervised training technique for a neural network; backpropagation training is frequently employed in conjunction with feedforward neural networks. In one aspect, random weights are assigned to all the inputs and activations in a neural network. In a further aspect, the inputs are then presented to the network and the output(s) are calculated using these random weights. In a still further aspect, the calculated outputs are compared with the expected outputs and error is calculated. In another aspect, the error that is calculated is used to update and/or adjust the weights in all layers of the neural network system. In yet another aspect, this process is repeated until a predetermined endpoint has been reached. In one aspect, the predetermined endpoint correlates to a total error level that has dropped below some target threshold. In an alternative aspect, the endpoint correlates to the completion of a certain number of “training cycles” (where a “training cycle” represents the analysis of a known sample by the neural network, the comparison of the expected value to the value actually obtained, the calculation of an error value, and the backwards adjustment of weights throughout the system). In a further aspect, the endpoint can include a combination of error level and number of training cycles. Without wishing to be bound by theory, a backpropagation technique allows the error of a neural network to be minimized by using the derivatives of the error function.

An “activation function” is a component of a neural network. An activation function is required to introduce non-linearity into the inputs of a layer in a neural network. An activation function scales a weighted signal using a scalar-to-scalar function. A variety of activation functions are contemplated herein.

In a further aspect, the activation function used in the neural network of the present invention is selected from the group including, but not limited to, bipolar functions, competitive functions, Elliott functions, Gaussian functions, hyperbolic tangent functions, identity functions, linear functions, linear combination functions, piecewise linear functions, sine wave functions, sigmoidal functions, SoftMax functions, step functions, tangential functions, and threshold functions. In one aspect, a sigmoidal function is used.

### Hardware Components of the Petroleum Detection System

In one aspect, described herein is a computer program product for determining the grade of petroleum in a sample, the computer program product comprising:

a tangible storage medium readable by a computer system and storing instructions for execution by the computer system for performing a method comprising:
a. determining a calibration curve that correlates the amount of fluorescence to the grade of petroleum the sample;
b. determining the amount of fluorescence produced by the petroleum in the sample;
c. comparing the amount of fluorescence produced by the petroleum in the sample to the calibration curve; and
d. calculating the grade of petroleum in a sample.

In another aspect, described herein is a system for determining the grade of petroleum in a sample on an instruction processing system, comprising:
a tangible storage medium readable by the instruction processing system and storing instructions for execution by the instruction processing system;
a calibration curve that correlates the amount of fluorescence to the grade of petroleum the sample;
a quantification module for obtaining the amount of fluorescence produced by the petroleum in the sample;
a comparison module for comparing the amount of fluorescence produced by the petroleum in the sample to the calibration curve; and
   a calculating module for determining the grade of petroleum in a sample.

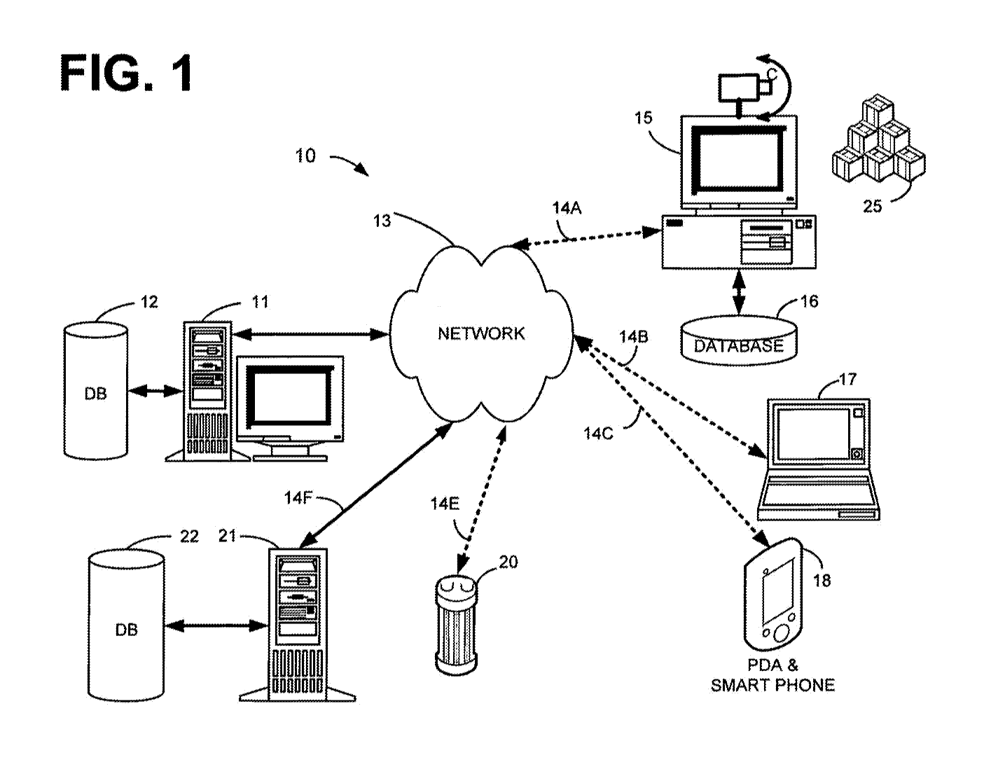

In FIGS. 1-5, like numerals illustrate like elements throughout the several views. FIG. 1 illustrates an example of the basic components of a petroleum detection system 10. The system 10 includes a server 11 and the remote devices 15, 17, and 18 that utilize the petroleum detection system.

Each remote device 15, 17, and 18 has applications and can have a local database 16. Server 11 contains applications, and a database 12 that can be accessed by remote devices 15, 17, and 18 via connections 14(A-C), respectively, over network 13. The server 11 runs administrative software for a computer network and controls access to itself and to database 12. The remote devices 15, 17, and 18 may access the database 12 over a network 13, including, but not limited to: the Internet, a local area network (LAN), a wide area network (WAN), via a telephone line using a modem (plain old telephone service or POTS), Bluetooth, WiFi, cellular, optical, satellite, RF, Ethernet, magnetic induction, coax, RS-485, and/or other like networks. The server 11 may also be connected to the local area network (LAN) within an organization (i.e., an oil refinery complex).

The remote devices 15, 17, and 18 may each be located at remote sites. Remote devices 15, 17, and 18 include, but are not limited to, PCs, workstations, laptops, netbooks and/or light notebook computers, pocket PCs, PDAs, pagers, WAP devices, non-WAP devices, cell phones, smart phones, palm devices, tablet computers, printing devices, and the like. Included with each of the remote devices 15, 17, and 18 is an ability to obtain images of the material being analyzed. In the remote device 15, there is a special camera 24 for capturing images of the sample being analyzed. In remote devices 17 and 18, there may be integrated cameras for acquiring images of the material being analyzed or the ability to download photographs of material being analyzed 25 in a digital form.

Thus, when a user of one of the remote devices 15, 17, and 18 desires to access petroleum data from the database 12 at the server 11, the remote devices 15, 17, and 18 communicate over the network 13 to access the server 11 and database 12.

A third-party vendor's computer system 21 and database 22 can be accessed by the petroleum detection system 100 on server 11 in order to access other analyzed materials and to provide analytics. Data that is obtained from the third party vendor's computer system 21 and database 22 can be stored on server 11 and database 12 in order to provide later access to the user on remote devices 15, 17, and 18. It is also contemplated that, for certain types of data, the remote devices 15, 17, and 18 can access the third party vendor's computer system 21 and database 22 directly using the network 13.

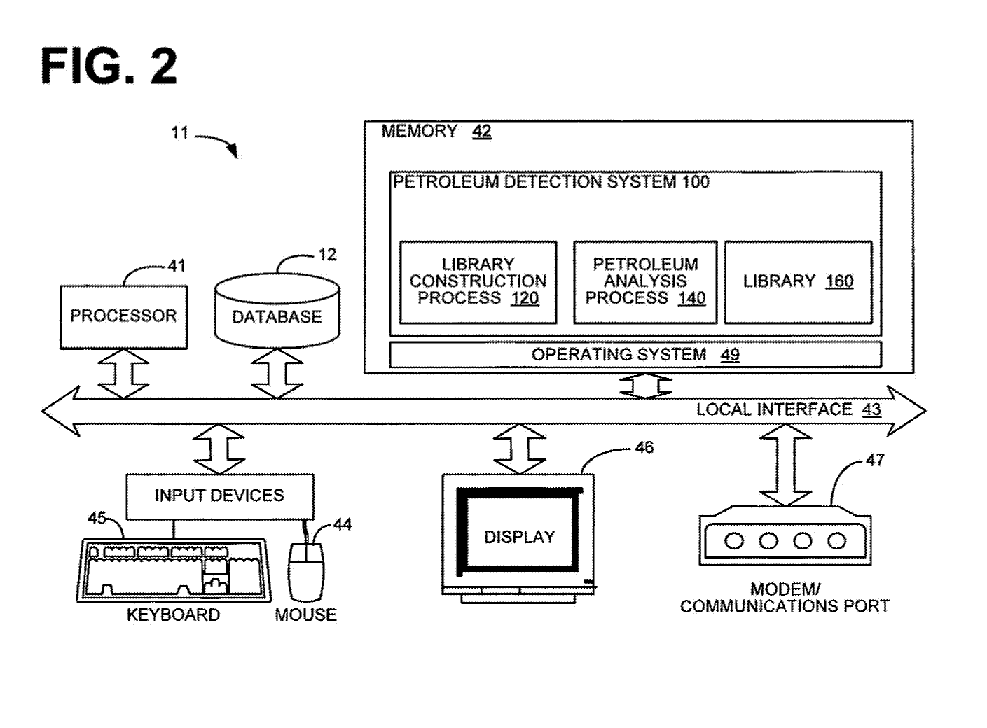

Illustrated in FIG. 2 is a block diagram demonstrating the example of server 11, as shown in FIG. 1, utilizing the petroleum detection system 100 described herein. Server 11 includes, but is not limited to, PCs, workstations, laptops, PDAs, tablet computers, and the like. The processing components of the third party vendor's computer system 21 and remote devices 15, 17, and 18 are similar to that of the description for the server 11 (FIG. 2).

Generally, in terms of hardware architecture, as shown in FIG. 2, the server 11 includes a processor 41, memory 42, and one or more input and/or output (I/O) devices (or peripherals) that are communicatively coupled via a local interface 43. The local interface 43 can be, for example, one or more buses or other wired or wireless connections, as is known in the art. The local interface 43 may have additional elements, which are omitted for simplicity, such as controllers, buffers (caches), drivers, repeaters, and receivers, to enable communications. Further, the local interface 43 may include address, control, and/or data connections to enable appropriate communications among the aforementioned components.

The processor 41 is a hardware device for executing software that can be stored in memory 42. The processor 41 can be virtually any custom-made or commercially available processor, a central processing unit (CPU), data signal processor (DSP), or an auxiliary processor among several processors associated with the server 11, and a semiconductor-based microprocessor (in the form of a microchip) or a macroprocessor. Examples of suitable commercially available microprocessors include, but are not limited to: an 80×86, Pentium, Core, or Celeron microprocessor from Intel Corporation, USA; a POWER or PowerPC microprocessor from IBM, USA; a SPARC microprocessor from Sun Microsystems, Inc., USA; a PA-series microprocessor from Hewlett-Packard, USA; an Athlon, Opteron, Sempron, or Turion microprocessor from AMD, USA; or a 68xxx series microprocessor from Motorola Corporation, USA.

The memory 42 can include any one or a combination of volatile memory elements including, but not limited to, random access memory (RAM, including dynamic random access memory, or DRAM, static random access memory, or SRAM, etc.) and nonvolatile memory elements including, but not limited to, read only memory (ROM, including erasable programmable read only memory, or EPROM, electronically erasable programmable read only memory, or EEPROM, programmable read only memory, or PROM, tape, compact disc read only memory, or CD-ROM, digital versatile disc read only memory, or DVD-ROM, disk, diskette, cartridge, cassette, or the like). Moreover, the memory 42 may incorporate electronic, magnetic, optical, and/or other types of storage media. Note that the memory 42 can have a distributed architecture, where various components are situated remotely from one another, but can be accessed by processor 41.

The software in memory 42 may include one or more separate programs, each of which includes an ordered listing of executable instructions for implementing logical functions. In the example illustrated in FIG. 2, the software in the memory 42 includes a suitable operating system (O/S) 49 and the petroleum detection system 100 described. As illustrated, the petroleum detection system of the present invention has numerous functional components including, but not limited to, the library construction process 120, petroleum analysis process 140, and library 160.

A non-exhaustive list of examples of suitable commercially-available operating systems 49 is as follows (a) a Windows operating system available from Microsoft Corporation; (b) a Netware operating system available from Novell, Inc.; (c) a Macintosh operating system available from Apple Computer, Inc.; (d) a UNIX operating system, available for purchase from many vendors including Hewlett-Packard, Sun Microsystems, Inc., and AT&T; (e) a LINUX operating system, available as open source software from a variety of locations on the internet; (f) a run time VxWorks operating system from WindRiver Systems, Inc.; or (g) an appliance-based operating system, such as one of those implemented in handheld computers, personal digital assistants (PDAs), smart phones, and/or tablet computers (e.g. Symbian OS available from Symbian, Inc.; PalmOS or webOS available from Hewlett-Packard; Windows Mobile, Windows CE, or Windows 8 available from Microsoft; BlackBerry OS available from Research in Motion, Ltd.; and iOS available from Apple Computer, Inc.).

The operating system 49 essentially controls the execution of other computer programs, such as the petroleum detection system 100, and provides scheduling, input-output control, file and data management, memory management, and communication control and related services. However, it is contemplated by the inventors that the petroleum detection system 100 is applicable on all other commercially available operating systems.

The petroleum detection system 100 may be a source program, executable program (object code), script, or any other entity that includes a set of instructions to be performed. When a source program, the program is usually translated via a compiler, assembler, interpreter, or the like, which may or may not be included within the memory 42, so as to operate properly in connection with the O/S 49. Furthermore, the petroleum detection system 100 can be written as (a) an object-oriented programming language, which has classes of data and methods, or (b) a procedure programming language, which has routines, subroutines, and/or functions including, but not limited to: C, C++, C#, Pascal, BASIC, API calls, HTML, XHTML, XML, ASP scripts, FORTRAN, COBOL, Perl, Java, ADA, .NET, Python, and the like.

The I/O devices may include input devices including, but not limited to, a mouse 44, keyboard 45, scanner (not shown), microphone (not shown), stylus (not shown), trackpad (not shown), touch screen (not shown), etc. Furthermore, the I/O devices may also include output devices including, but not limited to, a printer (not shown), display 46, etc. Finally, the I/O devices may further include devices that communicate both inputs and outputs including, but not limited to, a NIC or modulator/demodulator 47 (for accessing remote devices, other files, devices, systems, or a network), a radio frequency (RF) or other transceiver (not shown), a telephonic interface (not shown), a bridge (not shown), a router (not shown), etc.

If the server 11 is a PC, workstation, intelligent device or the like, the software in the memory 42 may further include a basic input output system (BIOS, omitted from drawings for simplicity). The BIOS is a set of essential software routines that initialize and test hardware at startup, start the O/S 49, and support the transfer of data among the hardware devices. The BIOS is stored in some type of read-only memory, such as ROM, PROM, EPROM, EEPROM, or the like, so that the BIOS can be executed when the server 11 is activated.

When the server 11 is in operation, the processor 41 is configured to execute software stored within the memory 42, to communicate data to and from the memory 42, and generally to control operations of the server 11 pursuant to the software. The petroleum detection system 100 and the O/S 49 are read, in whole or in part, by the processor 41, perhaps buffered within the processor 41, and then executed.

When the petroleum detection system 100 is implemented in software, as is shown in FIG. 2, it should be noted that the petroleum detection system 100 can be embodied in any computer-readable medium for use by or in connection with an instruction execution system, apparatus, or device, such as a computer-based system, processor-containing system, or other system that can fetch the instructions from the instruction execution system, apparatus, or device, and execute the instructions.

In the context of this document, a “computer-readable medium” can be any means that can store, communicate, propagate, or transport the program for use by or in connection with the instruction execution system, apparatus, or device. The computer readable medium can include, for example, an electronic, magnetic, optical, electromagnetic, infrared, or semiconductor system, apparatus, device, propagation medium or other physical device or means that can contain or store a computer program for use by or in connection with a computer-related system or method.

More specific examples of the computer-readable medium include, but are not limited to, the following: an electrical connection (electronic) having one or more wires, a portable computer diskette (magnetic or optical), a random access memory (RAM, electronic), a read-only memory (ROM, electronic), an erasable programmable read-only memory (EPROM, EEPROM, or Flash; all electronic), an optical fiber (optical), and a portable compact disc or digital versatile disc memory (CD-ROM, CD R/W, DVD-ROM, or DVD R/W; all optical). Note that the computer-readable medium could even be paper or another suitable medium, upon which the program is printed or punched (as in paper tape, punched cards, etc.), as the program can be electronically captured, via, for instance, optical scanning of the paper or other medium, then compiled, interpreted, or otherwise processed in a suitable manner if necessary, and then stored in a computer memory.

In an alternative embodiment, where the petroleum detection system 100 is implemented in hardware, the petroleum detection system 100 can be implemented with any one or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

The remote devices 15, 17, and 18 provide access to the petroleum detection system 100 on server 11 and database 12 using the remote device system, including, but not limited to, an internet browser. The information accessed in server 11 and database 12 can be provided in a number of different forms including, but not limited to ASCII data, web page data (i.e. HTML), XML, comma or tab delimited text, rich text format (RTF), Unicode text (including UTF8, UTF16, or UTF32), binary, or any other type of formatted data.

As illustrated, the remote devices 15, 17, 18, and 21 are similar to the description of the components for server 11 described with respect to FIG. 2. Hereinafter, the remote devices 15, 17, and 18 will be referred to as remote device 15 for the sake of brevity.

### Operation of the Petroleum Detection System as Performed by the Server

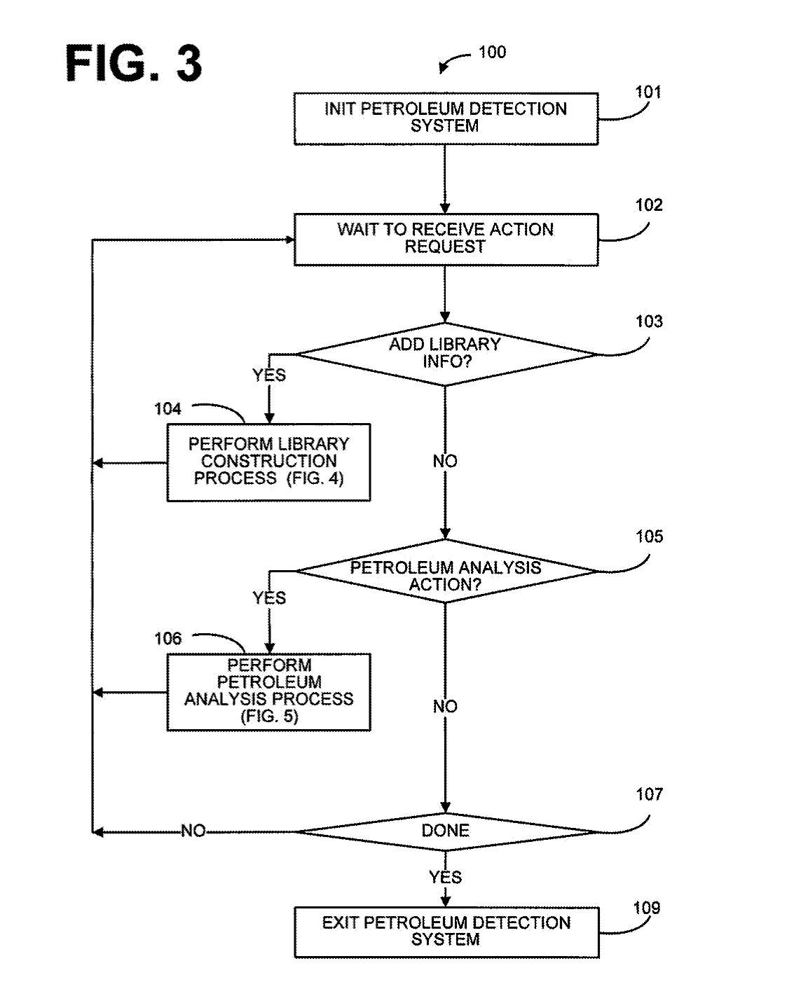

FIG. 3 is a flow chart illustrating an example of the operation of the petroleum detection system 100 described herein utilized by the server 11, as shown in FIG. 2. The petroleum detection system 100 quantifies the grade of petroleum in different media.

First, at step 101, the petroleum detection system 100 is initialized. This initialization includes the startup routines and processes embedded in the BIOS of the server 11. This initialization also includes the establishment of data values for particular data structures utilized in the petroleum detection system 100.

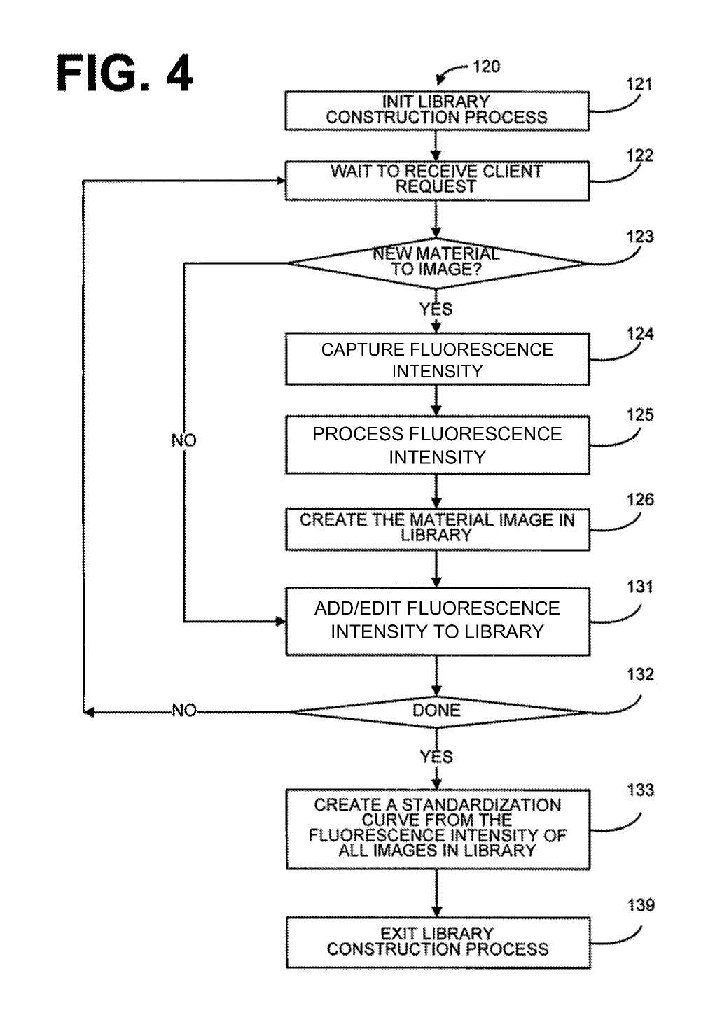

At step 102, the petroleum detection system 100 waits to receive an action request. Once an action request is received at step 102, it is determined if the action is to add a material sample to the library 160 at step 103. If it is determined that the action is not to add a new material sample to the library 160, then the petroleum detection system 100 skips step 105. However, if it is determined in step 103 that a new material sample is to be added to the library 160, then the petroleum detection system 100 performs the library construction process at step 104. The library construction process is herein defined in further detail with regard to FIG. 4. After performing the library construction process, the petroleum detection system 100 returns to step 102.

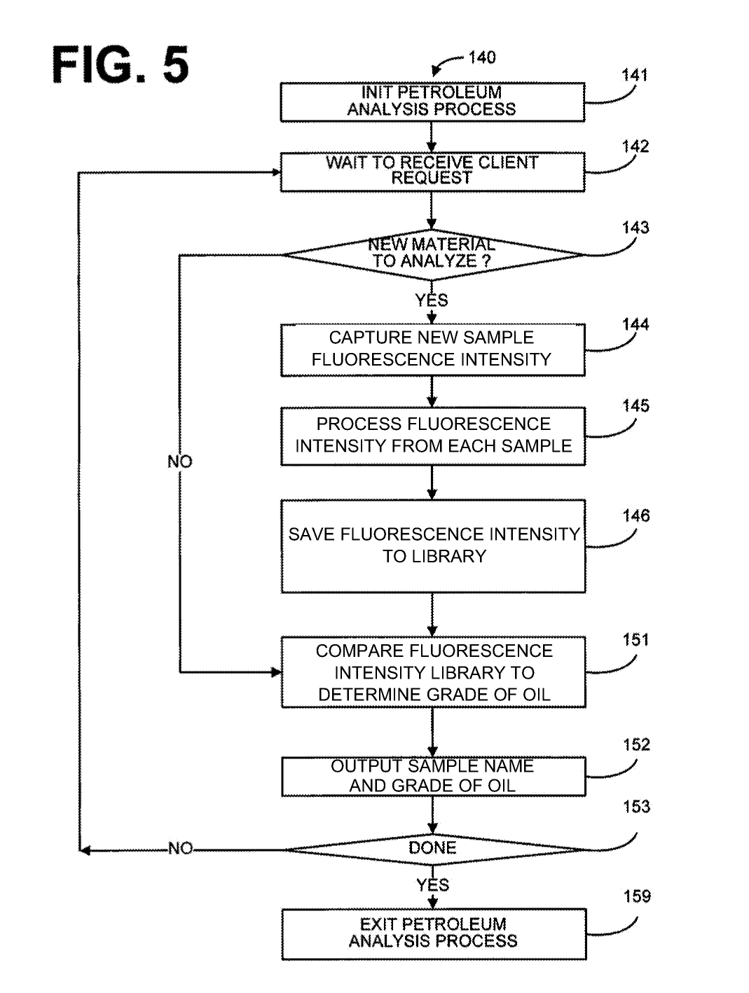

At step 105, it is determined if the action is a petroleum analysis action. If it is determined that the action is not a petroleum analysis action, then the petroleum detection system 100 skips step 107. However, if it is determined in step 105 that it is a petroleum analysis action, then the petroleum detection system 100 performs the petroleum analysis process at step 106. The petroleum analysis process is herein defined in further detail with regard to FIG. 5. After performing the petroleum analysis process, the petroleum detection system 100 returns to step 102.

At step 107, it is determined if the petroleum detection system 100 is to wait for an additional action request. If it is determined at step 107 that the petroleum detection system 100 is to wait to receive additional actions, then the petroleum detection system 100 returns to repeat steps 102 through 107. However, if it is determined at step 107 that there are no more actions to be received, then the petroleum detection system 100 exits at step 109.

FIG. 4 is a flow chart illustrating an example of the operation of the library construction process 120 on the server 11 that is utilized in the petroleum detection system 100, as shown in FIG. 2. The library construction process 120 establishes or modifies specific information residing in library 160 (FIG. 2). Once the new material information is placed in server 11, it is available for creating the standardization curve and performing petroleum analysis. A brief overview of one exemplary process is as follows: 1) wait to receive a client configure request; 2) determine if the material is a new material; 3) process fluorescence data from a sample; 4) upload new or modify existing material information from the local machine; and 5) done.

First, at step 121, the library construction process 120 is initialized. This initialization includes the startup routines and processes embedded in the BIOS of the server 11. The initialization step also includes the establishment of data values for particular data structures utilized in the library construction process 120.

At step 122, the library construction process 120 waits to receive a new client request. Once a new client request has been received, the library construction process 120 determines if the material is a new material to the petroleum detection system 100. If it is determined at step 123 that the material (i.e., fluorescence data from the spectrophotometer) is not a new material, then the library construction process 120 skips step 131 to enable the entry of new or the editing of existing material data. However, if it is determined at step 123 that the material is a new material, then the library construction process 120 captures the new material's image and fluorescence intensity at step 124. At step 125, the fluorescence intensity supplied by the spectrophotometer is processed along with its fluorescence intensity to create a library.

In one embodiment, step 125 in FIG. 4, a series of samples containing petroleum oil with known grades and amounts of transition metals that fluoresce when exposed to UV light can be prepared, where the fluorescence intensity can be captured by the spectrophotometer using an optical fiber and processed (steps 124 and 125). Each fluorescence intensity value for each sample can be input into a library. In step 126, the library can be developed using the ANSI C programming language (library “OpenGL”) and the Python programming language (library “Image”). The “OpenGL” and “Image” libraries permit the processing of pixels for each image for each sample. The library “OpenGL” is a library written in the C language, which connects the library 160 with the computational model. The data consists of each pixel of the processed image. The library “Image” permits the reading of the pixels of the image in order to create a plain file with information from each of the images.

At step 131, the library construction process 120 enables the addition of new image information or the editing of existing material information in the new material record. For example, in order to enhance the ability of the petroleum detection system 100 to determine petroleum grade in a sample, a fiber optic system can be used to optimize the standardization curve 133. Here, the fiber optic system is accurate than with respect to detecting and quantifying fluorescence produced by the oil present in the sample.

Referring to FIG. 1, the fiber optic sensor 20 detects fluorescence emitted by a sample having a known grade of oil. For example, a pure or neat petroleum oil sample having a known grade can be exposed to UV light, and the fluorescence emitted from the sample can be measured by the optical sensor and fed into the library. Here, a calibration curve correlating fluorescence intensity with the grade of the petroleum oil can be developed. In the case of a false positive (i.e., a soil sample with fluorescence but not containing petroleum), the difference in fluorescence between the real positive sample and the false positive sample determines the real value of the fluorescence to be included in the standardization curve. This has the effect of eliminating background noise. The fluorescence data is fed into the petroleum detection system 100 in order to optimize the standardization curve 133. More details regarding the use of the optical sensor or fiber are described below.

At step 132, it is determined whether the library construction process 120 is to wait for additional client requests. If it is determined at step 132 that the library construction process 120 is to wait for additional client requests, then the library construction process 120 returns to repeat steps 122 through 132. However, if it is determined at step 132 that there are no more client actions to be received, the library construction process 120 creates a current standardization curve from the fluorescence intensity of all analyzed material images in the library 160. After creating the new standardization curve, the library construction process 120 then exits at step 139.

FIG. 5 is a flow chart illustrating an example of the operation of the petroleum analysis process 140 on the server 11 that is utilized in the petroleum detection system 100, as shown in FIGS. 2 and 3. Once the new fluorescence data from the sample is introduced to server 11, it is available for petroleum analysis. A brief overview of one exemplary process is as follows: 1) wait to receive a client request for petroleum analysis; 2) determine if the material to be analyzed is new; 3) acquire a new sample spectrum and data regarding the new sample; 4) process fluorescence intensity at each wavelength in the new sample image; 5) create a new sample spectrum in library 160; 6) compare the new sample fluorescence spectrum to materials in library 160 to evaluate the grade of the petroleum in the sample; and 7) output the sample name, fluorescence intensity, and, grade of the petroleum.

At step 141, the petroleum analysis process 140 is initialized. This initialization includes the startup routines and processes embedded in the BIOS of the server 11. The initialization also includes the establishment of data values for particular data structures utilized in the petroleum analysis process 140.

At step 142, the petroleum analysis process 140 waits to receive a client transaction requesting sample analysis. Once a client transaction requesting sample analysis has been received, the petroleum analysis process 140 then determines if the material to be analyzed is a new sample at step 143. If the material to be analyzed is not a new sample, then the petroleum analysis process 140 skips step 151. However, if the material to be analyzed is a new sample, then the new sample's fluorescence emission spectrum is captured at step 144.

At step 145, the fluorescence intensity produced by the sample is processed. The optical sensor 20 can be used to measure the fluorescence intensity, where the fiber optic sensor 20 is preferred, due to its greater sensitivity. At step 146, a new record is created for the new sample in library 160 and information for the new sample is saved. This information saved includes, but is not limited to, excitation wavelength, emissions wavelength(s), fluorescence intensity, and the like.

At step 151, the new sample's fluorescence intensity is compared to data in library 160 in order to determine the grade of the petroleum in the new sample. This computer analysis is comparable to the computerized analysis of Pap smears and other tissue samples and cultures.

At step 152, the petroleum analysis process 140 outputs the sample name and fluorescence intensity of each material in the sample. At step 153, it is determined whether the petroleum analysis process 140 should wait for additional samples to be analyzed. If it is determined at step 153 that the petroleum analysis process 140 is to wait for additional client transactions, then the petroleum analysis process 140 returns to repeat steps 142 through 153. However, if it is determined at step 154 that there are no more samples to be analyzed, then the petroleum analysis process 140 then exits at step 159.

### Training the Petroleum Detection System

The petroleum detection system can be “trained” to correlate the amount of fluorescence to the density of the petroleum per the standards established by the American Petroleum Institute (API) and known as “API gravity.” For example, as discussed above, samples containing petroleum with a known API value can be exposed to UV light, and the fluorescence intensity can be measured by an optical fiber connected to a spectrophotometer. The fluorescence data can then be inputted into the library of the petroleum detection system.

As discussed above, samples of pure petroleum oil with a known grade can be exposed to UV light, and the fluorescence intensity can be measured and fed into the detection system.

In another aspect, samples containing different soil components containing oil with a known grade can also be used to train the petroleum detection system. In one aspect, reactors containing different soil samples and grades of oil can be used to train the petroleum detection system. The reactor can be filled with different types of soil sediments in order to simulate geological conditions at each site of interest. In one aspect, soil sample can be sandy soil, sludge, silt, clay sediment, sandy sediment, or any combination thereof. In one aspect, a reactor can be filled with soil having a specific constitution. Prior to introduction into the reactor, the soil is mixed with composition composed of a hydrocarbon having a known API grade and a digestion composition. The composition and function of the digestion composition is discussed below. In one aspect, the amount of hydrocarbon and digestion composition is sufficient to so that the soil is from 10% to 50%, 10% to 30%, or about 20% saturated with hydrocarbon. The saturated soil is introduced into the reactor, and the reactor is sealed.

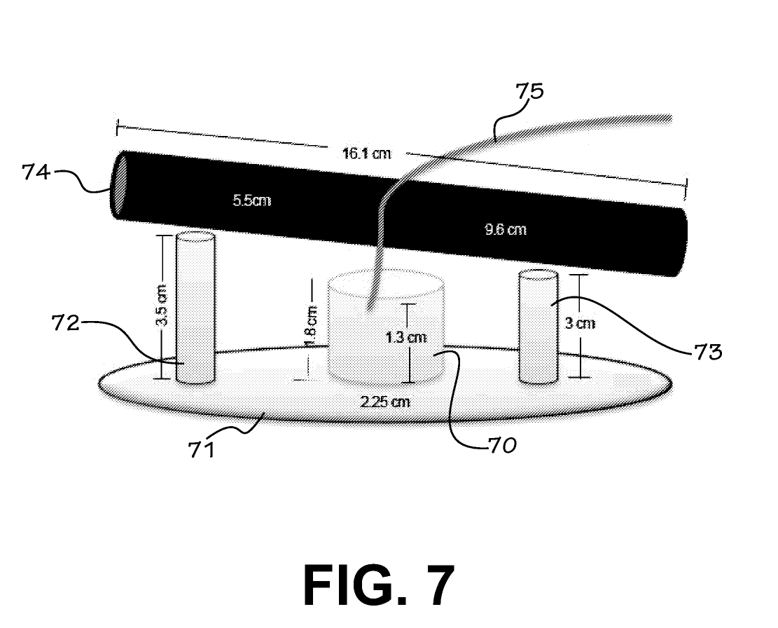

After a specified time, a specific amount of hydrocarbon (i.e., oil) in digestion composition is drained from the reactor. The sample is exposed to UV light, and the fluorescence is measured. FIG. 7 depicts one embodiment for performing this step. Referring to FIG. 7, the hydrocarbon sample is placed in sample container 70. The sample container 70 is mounted to base 71. Additionally, mounts 72 and 73 are attached to the base 71, which can receive and hold UV lamp 74. The height of the mounts 72 and 73 in FIG. 7 are different; however, the heights of the mounts can be the same or vary in order to optimize the exposure of the sample to UV light. Similarly, the numerical values shown in FIG. 7 are merely exemplary and can be adjusted as needed. Upon exposure of the sample to UV light, the sample will emit fluorescence. An optical fiber 75, which is connected to a spectrofluorimeter (not shown), is used to measure the fluorescence. In some aspects, the spectrofluorimeter may be a miniature or portable spectrofluorimeter such as the USB 2000+ manufactured by Ocean Optics (Dunedin, Fla.).

The optical fiber can be positioned such that it is contact with the hydrocarbon sample. In the alternative, the optical fiber can be positioned so that it is just above the hydrocarbon sample. The optical fiber with the spectrophotometer determines the fluorescence of the hydrocarbon sample with known API grade, and the data is fed into the neuronal network for training. The procedure above is repeated several times with different hydrocarbon samples having different API grades in order to further train the neuronal network. Additionally, the process described above can be used to calibrate the equipment or create a standard calibration curve for specific soil compositions. The Examples provide procedures for training the petroleum detection system to correlate fluorescence values to API gravities in order to assess the grade of oil present in the sample.

The digestion composition described above enhances the detection and quantification of the fluorescence produced by the hydrocarbon sample. Not wishing to be bound by theory, the digestion composition prevents the removal of impurities and other components that can reduce the fluorescence intensity produced by the sample upon exposure to UV light. By increasing the amount of fluorescence produced by the sample, more accurate readings and correlations between fluorescence and the grade of the oil are possible. Thus, the use of the digestion composition enhances the overall sensitivity of the methods described herein.

In one aspect, the digestion composition comprises a polysaccharide. As used herein, a “polysaccharide” is a carbohydrate molecule composed of repeated monomer units linked by glycosidic bonds. A polysaccharide may be linear or branched, and may contain only one type of monomer or may contain a mixture of monomers. A polysaccharide can contain substituents including, but not limited to, amino groups, acetyl groups, N-acetyl groups, hydroxyl groups, amide groups, alkylated amino groups, and/or alkylated hydroxyl groups.

In one aspect, the polysaccharide includes chitosan, glucosamine (GlcN), N-acetylglucosamine (NAG), or any combination thereof. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation in order to enhance tissue growth during culturing as well as metabolite production. In one aspect, the chitosan is from 60% to about 100%, 70% to 90%, 75% to 85%, or about 80% acetylated. Exemplary procedures for producing and isolating the chitosan are provided in the Examples. In one aspect, chitosan isolated from the shells of crab, shrimp, lobster, and/or krill is useful herein.

The molecular weight of the chitosan can vary, as well. For example, the chitosan comprises about 3, 4, 5, 6, 7, 8, 9, 10, 11, 12, 13, 14, 15, 16, 17, 18, 19, or 20 glucosamine units and/or N-acetylglucosamine units. In another aspect, the chitosan includes 5 to 7 glucosamine units and/or N-acetylglucosamine units. In one aspect, the chitosan is in a solution of water and acetic acid at less than 1% by weight, less than 0.75% by weight, less than 0.50% by weight, less than 0.25% by weight, or less than 0.10% by weight. The amount of polysaccharide present in the digestion composition can vary.

In another aspect, the digestion composition includes a polysaccharide and one or more organic solvents. In one aspect, the organic solvent comprises a linear or branched hydrocarbon, a halogenated hydrocarbon, or a mixture thereof. The selection of the organic solvent varies on the types and amounts of impurities present in the soil sample. For example, the soil sample may have impurities in the form of aromatic compound. Thus, it would be desirable to remove the petroleum oil from the sample without removing other impurities that can also emit fluorescence upon exposure to UV light. Examples of linear or branched hydrocarbons include, but are not limited to pentane, hexane, heptane, and branched isomers thereof. Examples of halogenated hydrocarbons include, but are not limited to, chloromethane, dichloromethane, and chloroform. In another aspect, the digestion composition includes chitosan, hexane, and dichloromethane. In one aspect, the digestion composition is from 1% to 10% by volume, 30% to 50% by volume hexane, and 30% to 50% by volume dichloromethane. In one aspect, 1 g to 10 g of soil sample can be contacted with a digestion composition composed of 1.5 to 15 mL of hexane, 1.5 mL to 15 mL of dichloromethane, 100 to 1,000 μL of polysaccharide solution, and 100 to 1,000 μL of dilute nitric acid.

### Testing of Samples Using the Petroleum Detection System

The petroleum detection system described herein is capable of determining the grade of petroleum in a sample. In one aspect, the method involves:
a. obtaining a sample comprising petroleum;
b. contacting the sample with a digestion composition;
c. filtering the sample in order to isolate a filtrate;
d. quantifying the amount of fluorescence produced by the filtrate;
e. comparing the amount of fluorescence in the filtrate to a standard curve, where the curve correlates the amount of fluorescence to the grade of petroleum; and
f. identifying the grade of petroleum in the sample.

In one aspect, a sample of known quantity is obtained and contacted with the digestion composition. The sample is mixed with the digestion composition to ensure that the digestions composition is evenly distributed throughout the sample. After the sample has been admixed with the digestion composition, the solution is filtered and the filtrate is collected by filtration. This step can be repeated multiple times depending upon the type and amounts of impurities present in the soil sample. The filtration step can be conducted using techniques known in the art. For example, filtration can be performed by the use of standard filter paper (e.g., Whatman size 0.4, 0.6, 0.8, or 1.0 microns depending upon the type of soil in the sample) or a glass-wool filter. After the filtrate has been isolated, a specified volume of filtrate is exposed to UV light and the fluorescence that is produced is measured and the value entered into the neuronal network where the grade of the oil present in the sample is determined In certain aspects, the petroleum detection system can be calibrated prior to testing of the samples. The use of the optical fiber as discussed above and depicted in FIG. 7 can be used here. The Examples provide exemplary procedures and for sample preparation and the determination of petroleum grade.

Any process descriptions or blocks in flow charts should be understood as representing modules, segments, or portions of code that include one or more executable instructions for implementing specific logical functions or steps in the process, and alternate implementations are included within the scope of the preferred embodiments of the present invention in which functions may be executed out of order from what is shown or discussed, including substantially concurrently or in reverse order, depending on the functionality involved, as would be understood by those reasonably skilled in the art of the present invention.

### EXAMPLES

The following examples are put forth so as to provide those of ordinary skill in the art with a complete disclosure and description of how the systems and methods described and claimed herein are constructed, operated, and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

### Neural Network Modeling

In this aspect, two neural networks were designed, one to detect the presence of petroleum in a sample and to determine the API gravity of the petroleum, and the other to detect and identify transition metals in a sample. Both neural networks received input in the form of a fluorescence emission spectrum, processed this input using an activation function and information stored in a library of known and/or previously-analyzed samples, and produced output that could be correlated to the grade of petroleum or the identity of transition metals in a sample, respectively. The “Encog” machine learning framework (available from Heaton Research, Chesterfield, Mo.) is used to implement the neural networks described herein; use of Encog allows for the saving and reuse of training data.

### Detection of the Presence of Petroleum in a Sample and Determination of the API Grade of the Petroleum

The neural network model to detect the presence and determine the API gravity of petroleum was designed as follows:

An input vector with a size of 400 units of type double was defined. Each position of the vector represented the intensity of a particular wavelength. Intensities of wavelengths ranging from about 300 to about 700 were used; outside this range, a low signal-to-noise ratio prevented useful analysis. An output vector with a size of 1 unit of type double was also defined. The only position in the vector contained a value which represented the API gravity of the sample. Finally, a hidden layer vector of size 350 units of type double was defined.

A sigmoidal activation function of the following form was used:

$$f ( x ) = \frac{1}{1 + {e}^{- x}}$$

This function has a maximum value of 1 and a minimum value of 0 and is differentiable throughout its domain (−∝,+∞). Sigmoidal functions are convenient normalization functions and are the most common type of activation function employed in neural network models. 10,000 training cycles using a supervised training algorithm were performed to obtain error of 1×10⁻¹⁰.

Data was normalized to have values between 0 and 1. The following equation was used, with a maximum value of 18,000 and a minimum value of 0:

$$Normalized Value = \frac{Maximum Value - Real Value}{Maximum Value - Minimum Value}$$

Use of this equation enabled the assignment of a unique output value of between 0 and 1, inclusive, for all data entered.

Detection of the Presence and/or Identity of Transition Metals in a Sample

The neural network model to detect the presence and determine the identity of transition metals was designed as follows:

An input vector with a size of 400 units of type double was defined. Each position of the vector represented the intensity of a particular wavelength. Intensities of wavelengths ranging from about 300 to about 700 were used; outside this range, a low signal-to-noise ratio prevented useful analysis. An output vector with a size of 2 units of type double was also defined. The first position in the vector represents vanadium and has a value of 0 if vanadium is absent and 1 if vanadium is present. The second position in the vector represents nickel and has a value of 0 if nickel is absent and 1 if nickel is present. Finally, a hidden layer vector of size 350 units of type double was defined.

A sigmoidal activation function was again used. This function has a maximum value of 1 and a minimum value of 0 and is differentiable throughout its domain (−∞,+∞). Sigmoidal functions are convenient normalization functions and are the most common type of activation function employed in neural network models. 10,000 training cycles using a supervised training algorithm were performed to obtain error of 1×10⁻¹⁰.

Data was normalized to have values between 0 and 1, using the same equation as was previously described, with a maximum value of 18,000 and a minimum value of 0.

Use of this equation enabled the assignment of a unique output value of 0 or 1 for all data entered.

### Feedforward Phase

The feedforward phase of the neural networks described herein began when an input pattern was fed into the input layer of the neural networks. The input units are set to the corresponding elements of the input pattern and the value or level of activation of the output of the first (input) layer is calculated. The same process occurs in each subsequent layer of the feedforward neural network model used herein.

Inputs to be passed to subsequent layers are calculated using the following equation:

Sⱼ=Σᵢ(Aᵢ×Wⱼᵢ)

where Sj is the sum of all relevant products of weights and outputs from the previous layer i, Wᵢⱼ represents the relevant weights connecting layer i with layer j, Aᵢ represents the activations of the nodes in the previous layer i, Aⱼ is the activation of the node at hand, and f is the activation function.

Output of the neural network, more generally, is defined by the following equation:

Output=f(Sⱼ)

Where the function f is a generic threshold function which can be a sigmoidal function. The value of the output of unit j is transmitted over all the output connections of said unit.

### Backpropagation

Each unit of the output layer produces a real number as output and adjusting the weights of the inputs is simple because, during the training process, known samples were used. Based on the known (desired) result and the actual result produced by the neural network, error was calculated for each unit in the output layer according to the following equation:

δⱼ=(tⱼ−Aⱼ)×f′(Sⱼ)

Where δⱼ is an error value, tⱼ is the desired output value, Aⱼ the value contained within output neuron j, and f′ the derivative of threshold function f. Based on the calculated error, the system adjusts the weights in the neurons according to the following equation; the cycle is repeated until the desired error threshold has been reached.

Δwᵢⱼ=η×δⱼ×Aᵢ

Where η is the learning coefficient, usually a value between 0.25 and 0.75 that reflects the degree of network learning, Δw is a change in weights between layers i and j, and the other variables have been defined previously. The coefficient η may have a high initial value and become progressively lower during the training session with the aim of achieving better learning.

### Evaluation of Training Data

After the training process, training evaluation is conducted. 100 total samples were used to test the performance of the system. 50 samples were used to train the system, and the other 50 were used to test and verify the correct operation of the system.

### Evaluation of Unknown Samples

50 petroleum samples with different API gravities were presented to the neural networks described herein. These samples had not previously been presented to the system. Predicted API gravities had an error rate of about 5% of the actual values; the error for unknown samples was higher as these samples had never been presented to the computational model during the training process.

### Libraries Created

Several libraries were created during the process of setting up and training the neural networks described herein. Short descriptions of these libraries follow.

The library “Cargardatosespectro” (“load spectrum data”) loaded the data to be processed to create training maps. 30 fluorescence spectra for each training sample were stored in a directory and a function in this library created a table containing the spectral data and passed them on to functions from the “Maps” library.

The library “Maps” was responsible for creating training maps to feed to the neural networks for both API gravity and transition metal detection. This library read 30 files saved in a directory and created a single input vector of type double and size 400 (this information is obtained from fluorescence spectra by calling a function). When the information was loaded, negative values were deleted. The single input vector was created by averaging 30 individual fluorescence spectra for a sample.

The library “redneuronal_api” was responsible for creating the neural network that trained and tested the API gravity of a given sample. This library allowed the entry of input and output values from training maps; these values were then normalized. Functions in this library were also responsible for training and creating persistence files for the neural network.

The library “redneuronal_metales” was responsible for creating the neural network that trained and tested the presence of transition metals in a given sample. This library allowed the entry of input and output values from training maps; these values were then normalized. Functions in this library were also responsible for training and creating persistence files for the neural network.

The library “TimesSeriechardemo” contains functions for graphical analysis of the data being analyzed.

The library “encog” has been discussed previously. The library “JFreeChart” is an open source library for graphics in the Java programming language. This library enables developers to easily handle high quality graphics for the purpose of developing desktop applications.

### Chitosan

Shrimp shells were subjected to a process of washing and drying in an oven and they were milled so that smaller particle size is obtained. Once the shrimp shells are in the form of powder, the powder was subjected to deproteinization using sodium hydroxide 10% w/v in a ratio of 1/5 per volume with an electric shaker (250 RPM). After two hours at a temperature of 80° C., the powder was filtered, washed with water, then the deproteinization process repeated with 10% NaOH.

Once deproteinized, the product was washed with water until neutral. Then product was treated with hydrochloric acid 5% v/v (ratio of 1/5 per volume product to acid) and stirred for two hours at 250 rpm at room temperature. The product was washed, and the process above was repeated with HCl. The product was washed with water to remove any residual acid.

The neutralized product was dried for 18 hours in an oven at 100° C. up to 3% humidity and a 20% performance compound 1. The dried product was subjected to deacetylation using a solution of NaOH 50% with vigorous stirring at 250 rpm for 4 hours at a temperature of 100° C. The ratio of product to NaOH was 1/8 weight to volume. The NaOH solution was removed and the solid washed to remove any base. The product was dried resulting in a performance of 65% with respect to Compound 1. The final chitosan product had a moisture percentage of 1%.

### Preparation of Reactors

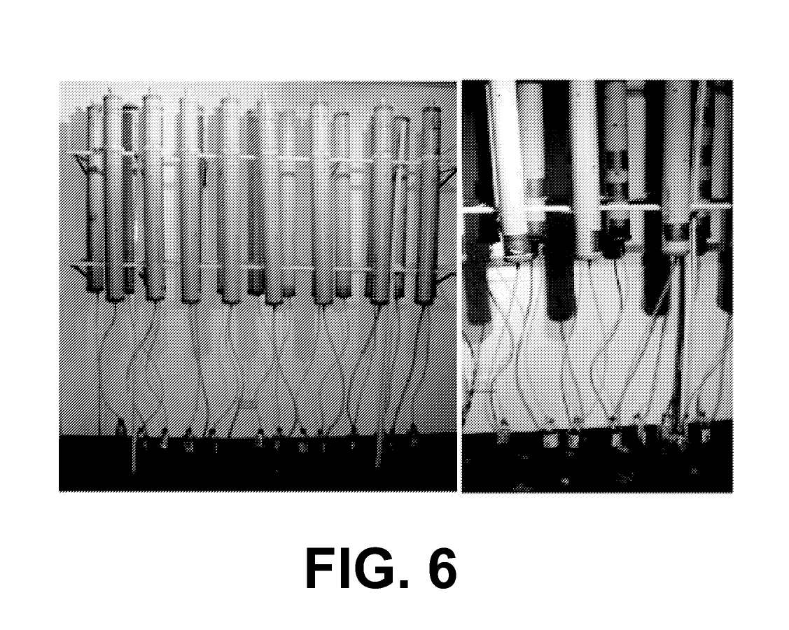

To validate the tests used to assess different types of petroleum, reactors were constructed to simulate the actual behavior of petroleum in the Earth. FIG. 6 depicts exemplary reactors that can be used. Reactor construction proceeded as follows:

Reactors were 30 cm long and 15 cm in diameter. Each reactor contained three orifices where samples could be drawn from the reactors. Reactors were constructed in modular form to allow joining with other reactors so as to lengthen or expand in height the size of the reactors. For example, joining two reactors would create a new reactor that was 60 cm long with six sampling orifices. A distance of 3 cm typically existed between sampling orifices, and sampling orifices had diameters of 1 cm.

Reactors were filled with 3500 g of natural soil. This soil was saturated with 112.5 mL hexane, 112.5 mL dichloromethane, and 45 mL of petroleum hydrocarbons, which resulted in approximately 20% saturation of the soil with hydrocarbons.

After saturating the soil as described above, the reactors were covered and allowed to drain for six days. Next, a total of 18 samples were measured from each reactor (samples were taken from each of 6 different sampling orifices at 3 different times, for a total of 18).

### Sample Preparation

After samples were removed from the reactors, they were prepared for analysis according to the following protocol. A 1 g sub-sample was taken from each sample. 1.5 mL of hexane, 1.5 mL of dichloromethane, 100 μL of polysaccharide solution, and 100 μL of dilute nitric acid were added to each sample. Samples were then homogenized and filtered into a test tube prior to measurement. In another experiment, when ten times the amount of starting materials was used, more accurate results with respect to the determination of the petroleum grade were achieved.

### Results

API gravity of each sample was evaluated using a densitometer and ASTM method D5002 at a laboratory certified for the identification of petroleum. Results for some test samples are presented in Table 1.

TABLE 1
Theoretical Values, ASTM D5002 Values, and Experimental Values of
API Gravity for Known Samples

| | Theoretical | API Gravity | API Gravity |
|---|---|---|---|
| Sample | Value | (ASTM D5002) | (Experimental) |
| 1 | 9 | 9.9 | 9 |
| 2 | 11 | 11.06 | 11.3 |
| 3 | 45 | 44.19 | 45 |

The fiber optic sensor was immersed in the homogenized sample. A shadowless UV lamp was placed over the sample. Thirty fluorescence measurements were taken for each sample tested, and the results are presented in Table 2 below:

TABLE 2
Increased Fluorescence Intensity Using a New Measurement System
Design

| Sample | Fluorescence | | |
|---|---|---|---|
| API | Intensity | Standard | Number of |
| Gravity | (New Design) | Deviation | Measurements |
| 0 | 1095 | ±148 | 30 |
| 8 | 4941 | ±320 | 30 |
| 11 | 7885 | ±187 | 30 |
| 45 | 15567.8 | ±422 | 30 |

### Quantification of Fluorescence to Determine Petroleum API Gravity

A classification method for petroleum samples was developed using the neural networks described herein. This computational method predicted the output ranges which delineate the different API petroleum gravities. Table 3 includes the average neural network output value for each API petroleum gravity.

TABLE 3
Average Neural Network Output for Different Grades of Petroleum

| API Petroleum Gravity | Average Neural Network Output |
|---|---|
| 0 | 3537.531 |
| 1 | 3537.591 |
| 2 | 3537.670 |
| 3 | 3537.771 |
| 4 | 3537.901 |
| 5 | 3538.069 |
| 6 | 3538.286 |
| 7 | 3538.566 |
| 8 | 3538.928 |
| 9 | 3539.395 |
| 10 | 3539.997 |
| 11 | 3540.776 |
| 12 | 3541.780 |
| 13 | 3543.077 |
| 14 | 3544.752 |
| 15 | 3546.915 |
| 16 | 3549.708 |
| 17 | 3553.316 |
| 18 | 3557.977 |
| 19 | 3564.000 |
| 20 | 3571.786 |
| 21 | 3581.855 |
| 22 | 3594.882 |
| 23 | 3611.748 |
| 24 | 3633.603 |
| 25 | 3661.952 |
| 26 | 3698.774 |
| 27 | 3746.681 |
| 28 | 3809.137 |
| 29 | 3890.761 |
| 30 | 3997.745 |
| 31 | 4138.433 |
| 32 | 4324.099 |
| 33 | 4569.956 |
| 34 | 4896.352 |
| 35 | 5329.858 |
| 36 | 5903.472 |
| 37 | 6654.129 |
| 38 | 7614.492 |
| 39 | 8795.935 |
| 40 | 10164.703 |
| 41 | 11625.781 |
| 42 | 13038.214 |
| 43 | 14266.926 |
| 44 | 15235.938 |
| 45 | 15941.869 |
| 46 | 16428.601 |
| 47 | 16753.886 |
| 48 | 16968.777 |

1. A method for determining the grade of petroleum in a sample, the method comprising:
a. obtaining a sample comprising petroleum;
b. contacting the sample with a digestion composition;
c. filtering the sample in order to isolate a filtrate;
d. quantifying the amount of fluorescence produced by the filtrate;
e. comparing the amount of fluorescence in the filtrate to a standard curve, where the curve correlates the amount of fluorescence to the grade of petroleum; and
f. identifying the grade of petroleum in the sample.

2. The method of claim 1, wherein the digestion composition comprises a polysaccharide.

3. The method of claim 1, wherein the digestion composition comprises chitosan.

4. The method of claim 2, wherein the digestion composition comprises one or more organic solvents.

5. The method of claim 4, wherein the organic solvent comprises a linear or branched hydrocarbon, a halogenated hydrocarbon, or a mixture thereof.

6. The method of claim 1, wherein the digestion composition comprises chitosan, hexane, and dichloromethane.

7. The method of claim 1 wherein steps (e) and (f) are performed by a computer program product for execution on an instruction processing system, comprising a tangible storage medium readable by the instruction processing system and storing instructions for execution by the instruction processing system.

8. The method of claim 1, wherein the sample comprises a soil sample.

9. The method of claim 8, wherein the sample comprises sandy soil, sludge, clay sediment, sandy sediment, or any combination thereof.

10. The method of claim 1, wherein the sample comprises of petroleum comprises vanadium, nickel, iron, copper, or any combination thereof.

11. The method of claim 1, wherein step (d) comprises (1) exposing the sample to UV light and (2) quantifying the amount of fluorescence by an optical fiber sensor.

12. A computer program product for determining the grade of petroleum in a sample, the computer program product comprising:
a tangible storage medium readable by a computer system and storing instructions for execution by the computer system for performing a method comprising:
a. determining a calibration curve that correlates the amount of fluorescence to the grade of petroleum the sample;
b. determining the amount of fluorescence produced by the petroleum in the sample;
c. comparing the amount of fluorescence produced by the petroleum in the sample to the calibration curve; and
d. calculating the grade of petroleum in a sample.

13. A system for determining the grade of petroleum in a sample on an instruction processing system, comprising:
a tangible storage medium readable by the instruction processing system and storing instructions for execution by the instruction processing system;
a calibration curve that correlates the amount of fluorescence to the grade of petroleum the sample;
a quantification module for obtaining the amount of fluorescence produced by the petroleum in the sample;
a comparison module for comparing the amount of fluorescence produced by the petroleum in the sample to the calibration curve; and

[symbol]

14. a calculating module for determining the grade of petroleum in a sample.
